(19) United States
(12) Patent Application Publication
(10) Pub. No.: US 2011/0124565 A1
(43) Pub. Date: May 26, 2011

(54) Modified Coagulation Factor VIII With Enhanced Stability and Its Derivatives

(76) Inventors: Hans-Peter Hauser, Marburg (DE); Thomas Weimer, Gladenbach (DE); Jean-Luc Plantier, Gringy (FR); Marie-Hélène Rodriguez, Nivolas Vermelie (FR); Claude Négrier, Irigny (FR)

(21) Appl. No.: 11/918,280

(22) PCT Filed: Apr. 10, 2006
(86) PCT No.: PCT/EP2006/003249

§ 371 Date: Oct. 11, 2007

(30) Foreign Application Priority Data
Apr. 14, 2005 (EP) 05008152.0

(51) Int. Cl. A61K 38/37; C07K 14/755; C07H 21/00; C12N 15/63; C12N 5/10; C12P 21/00; A61K 31/7088; A61P 7/04

(52) U.S. Cl. 514/14.1; 530/383; 536/23.5; 435/320.1; 435/325; 435/69.6; 514/44 R

(57) ABSTRACT

The present invention relates to modified nucleic acid sequences coding for coagulation factors, in particular human Factor VIII and their derivatives with improved stability, recombinant expression vectors containing such nucleic acid sequences, host cells transformed with such recombinant expression vectors, recombinant polypeptides and derivatives which do have biological activities of the unmodified wild type protein but having improved stability and processes for the manufacture of such recombinant proteins and their derivatives. The invention also covers a transfer vector for use in human gene therapy, which comprises modified DNA sequences.

The present invention relates to modified nucleic acid sequences coding for coagulation factors, in particular human Factor VIII and their derivatives with improved stability, recombinant expression vectors containing such nucleic acid sequences, host cells transformed with such recombinant expression vectors, recombinant polypeptides and derivatives which do have biological activities of the unmodified wild type protein but having improved stability and processes for the manufacture of such recombinant proteins and their derivatives. The invention also relates to a transfer vector for use in human gene therapy, which comprises such modified nucleic acid sequences.

Classic hemophilia or hemophilia A is an inherited bleeding disorder. It results from a chromosome X-linked deficiency of blood coagulation Factor VIII, and affects almost exclusively males with an incidence of between one and two individuals per 10.000. The X-chromosome defect is transmitted by female carriers who are not themselves hemophiliacs. The clinical manifestation of hemophilia A is an increased bleeding tendency. Before treatment with Factor VIII concentrates was introduced the mean life span for a person with severe hemophilia was less than 20 years. The use of concentrates of Factor VIII from plasma has considerably improved the situation for the hemophilia patients increasing the mean life span extensively, giving most of them the possibility to live a more or less normal life. However, there have been certain problems with the plasma derived concentrates and their use, the most serious of which have been the transmission of viruses. So far, viruses causing AIDS, hepatitis B, and non-A non-B hepatitis have hit the population seriously. Since then different virus inactivation methods and new highly purified Factor VIII concentrates have recently been developed which established a very high safety standard also for plasma derived Factor VIII.

The cloning of the cDNA for Factor VIII (Wood, W. I., et al. (1984) Nature 312, 330-336; Vehar, G. A., et al. (1984) Nature 312, 337-342) made it possible to express Factor VIII recombinantly leading to the development of several recombinant Factor VIII products, which were approved by the regulatory authorities between 1992 and 2003. The fact that the central B domain of the Factor VIII polypeptide chain residing between amino acids Arg-740 and Glu-1649 does not seem to be necessary for full biological activity has also led to the development of a B domain deleted Factor VIII.

The mature Factor VIII molecule consists of 2332 amino acids which can be grouped into three homologous A domains, two homologous C domains and a B Domain which are arranged in the order: A1-A2-B-A3-C1-C2. The complete amino acid sequence of mature human Factor VIII is shown in SEQ ID NO:2. During its secretion into plasma Factor VIII is processed intracellularly into a series of metal-ion linked heterodimers as single chain Factor VIII is cleaved at the B-A3 boundary and at different sites within the B-domain. This processing leads to a heavy chain consisting of the A1, the A2 and various parts of the B-domain which has a molecular size ranging from 90 kDa to 200 kDa. The heavy chains are bound via a metal ion to the light chain, which consists of the A3, the C1 and the C2 domain (Saenko et al. 2002). In plasma this heterodimeric Factor VIII binds with high affinity to von Willebrand Factor, which protects it from premature catabolism. The half-life of non-activated Factor VIII bound to VWF is about 12 hours in plasma.

During the blood coagulation process Factor VIII is activated via proteolytic cleavage by FXa and thrombin at amino acids Arg372 and Arg740 within the heavy chain and at Arg1689 in the light chain resulting in the release of von Willebrand Factor and generating the activated Factor VIII heterotrimer which will form the tenase complex on phospholipid surfaces with FIXa and FX provided that Ca²⁺ is present. The heterotrimer consists of the A1 domain, a 50 kDa fragment, the A2 domain a 43 kDa fragment and the light chain (A3-C1-C2), a 73 kDa fragment. Thus the active form of Factor VIII (Factor Villa) consists of an A1-subunit associated through the divalent metal ion linkage to a thrombin-cleaved A3-C1-C2 light chain and a free A2 subunit relatively loosely associated with the A1 and the A3 domain.

To avoid excessive and disseminated coagulation, Factor VIIIa must be inactivated soon after activation. The inactivation of Factor VIIIa via activated Protein C (APC) by cleavage at Arg336 and Arg562 is not considered to be the rate-limiting step. It is rather the dissociation of the non-covalently attached A2 subunit from the heterotrimer which is thought to be the rate limiting step in Factor VIIIa inactivation after thrombin activation (Fay, P. J. et al, J. Biol. Chem. 266: 8957 (1991), Fay P J & Smudzin T M, J. Biol. Chem. 267: 13246-50 (1992)). This is a rapid process, which explains the short half-life of Factor VIIIa in plasma, which is only 2.1 minutes (Saenko et al., Vox Sang. 83: 89-96 (2002)). Therefore increasing the affinity of the A2 domain to the A1/A3-C1-C2 heterodimer would prolong the half-life of Factor Villa and a Factor VIII with increased haemostatic activity would be obtained.

In severe hemophilia A patients undergoing prophylactic treatment Factor VIII has to be administered i.v. about 3 times per week due to the short plasma half life of Factor VIII of about 12 hours. Each i.v. administration is cumbersome, associated with pain and entails the risk of an infection especially as this is mostly done in home treatment by the patients themselves or by the parents of children being diagnosed for hemophilia A.

It would thus be highly desirable to create a Factor VIII with increased functional half-life allowing the manufacturing of pharmaceutical compositions containing Factor VIII, which have to be administered less frequently.

Several attempts have been made to prolong the half-life of non-activated Factor VIII either by reducing its interaction with cellular receptors (WO 03/093313A2, WO 02/060951A2), by covalently attaching polymers to Factor VIII (WO 94/15625, WO 97/11957 and U.S. Pat. No. 4,970,300) or by encapsulation of Factor VIII (WO 99/55306).

In WO 97/03193 it was speculated that the introduction of novel metal binding sites could stabilize Factor VIII and in particular mutants in which His or Met is substituted for any of Phe652, Tyr1786, Lys1818, Asp1840 and/or Asn1864. However no rationale was provided how to determine the success meaning the stabilization resulting from such modifications nor a rationale why the proposed amino acids were chosen.

Another approach has been made in creating a Factor VIIIa which is inactivation resistant by covalently attaching the A2 domain to the A3 domain and by mutating the APC cleavage sites (Pipe and Kaufman, PNAS, (1997) 94:11851-11856, WO 97/40145 and WO 03/087355.). This genetic construct was also used to produce transgenic animals as described in WO 02/072023A2. This variant showed still 38% of its peak activity 4 h after thrombin activation. This variant however lacks the VWF binding domain as by fusing the A2 to the A3 domain this domain was deleted. As VWF binding significantly prolongs half-life of FVIII in vivo, it is to be expected that half-life of the non-activated form of IR8 is compromised. The inventors themselves recognized this and try by adding an antibody to compositions of the modified FVIII to overcome this problem.

Gale et al. (Protein Science (2002), 11:2091-2101) published the stabilization of FV by covalently attaching the A3 domain to the A2 domain. They identified two neighbouring amino acids according to structural predictions, one on the A2 domain and the other being located on the A3 domain, and replaced these two amino acids with cysteine residues, which formed a disulfide bridge during export into the endoplasmatic reticulum. The same approach was used to covalently attach via disulfide bridges the A2 to the A3 domain of Factor VIII (WO 02/103024A2). Such covalently attached Factor VIII mutants retained about 90% of their initial highest activity for 40 minutes after activation whereas the activity of wild type Factor VIII quickly went down to 10% of its initial highest activity. The Factor VIII mutants retained their 90% activity for additional 3 h without any further loss of activity (Gale et al., J. Thromb. Haemost. (2003), 1:1966-1971). It remains to be seen whether these FVIII variants will also be stable after thrombin activation in vivo and whether it will not be thrombogenic as it has recently been shown that constitutively high levels of Factor VIII might constitute a risk factor for thromboembolism (Kyrle 2003, Hamostasiologie 1: p. 41-57).

Hence, there is an ongoing need to develop modified blood coagulation factors which exhibit favourable properties.

Previously it was thought that thrombin mediated cleavage at Arg372 is a prerequisite for FVIII activation, which was supported e.g. by the generation of inactive FVIII variants when Arg372 was replaced with Ile (Pittman (1988), PNAS 85:2429-2433). In the present invention it has been surprisingly found that a stabilized FVIII variant can be obtained which is biologically active after thrombin activation by introducing mutations that are characterised in that they prevent thrombin cleavage between the A1 and the A2 domain of FVIII and therefore keep the A2 domain covalently attached to the A1 domain after thrombin activation.

In a first aspect, the invention therefore relates to modified FVIII variants, characterised by a modification that prevents thrombin cleavage between the A1 and the A2 domain of FVIII. Therefore the A2 domain remains covalently attached to the A1 domain after thrombin activation and these FVIII variants remain functionally active and display prolonged functional half-life after activation by thrombin to FVIIIa. The FVIII variants of the invention have an inactivated thrombin cleavage site at R372, which can by way of a nonlimiting example be realized by mutating R372 into A372. A peptidic linker sequence may be introduced between the A1 and the A2 domain, which should be flexible and not immunogenic (Robinson et al.; PNAS (1998), Vol 95, p 5929). In a preferred embodiment of the invention the peptidic linkers replace Val374 (Seq ID No 2) with Gly preceded N-terminally to said Gly by multimers of the amino acid sequence GlyGlySer or GlyGlySerSer or any combination thereof, in a particularly preferred embodiment the peptidic linker consists of 80 to 120 amino acids, even more preferred is a peptidic linker of 90 to 110 amino, most preferred is a peptidic linker of 99 amino acids.

FVIII from all vertebrate species can be stabilized based on the present invention. Of particular interest are human and porcine modified FVIII variants. Also chimeric FVIII variants from different species are one aspect of the invention, e.g. human/porcine (U.S. Pat. No. 5,364,771) or human/murine chimera.

Also chimeric molecules of FV and FVIII are another aspect of the invention (Marquette et al. 1995, JBC, 270:10297-10303, Oertel et al. 1996, Thromb. Haemost. 75:36-44).

The FVIII variants can be based on wild type FVIII or on FVIII variants in which the B-domain is partially or completely deleted and is optionally replaced by a linker.

The terms “blood coagulation Factor VIII”, “Factor VIII” and FVIII″ are used interchangeably herein. “Blood coagulation Factor VIII” includes derivatives of wild type blood coagulation Factor VIII having the procoagulant activity of wild type blood coagulation Factor VIII. Derivatives may have deletions, insertions and/or additions compared with the amino acid sequence of wild type Factor VIII. As non-limiting examples, Factor VIII molecules include full-length recombinant Factor VIII, B domain deleted Factor VIII (Pittman 1993, Blood 81:2925-2935), Factor VIII mutants preventing or reducing APC cleavage (Amano 1998, Thromb. Haemost. 79:557-563), Factor VIII mutants further stabilizing the A2 domain (WO 97/40145), FVIII mutants resulting in increased expression (Swaroop et al. 1997, JBC 272:24121-24124), Factor VIII mutants reducing its immunogenicity (Lollar 1999 Thromb. Haemost. 82:505-508), FVIII reconstituted from differently expressed heavy and light chains (Oh et al. 1999, Exp. Mol. Med. 31:95-100), FVIII mutants reducing binding to receptors leading to catabolism of FVIII like HSPG (heparan sulfate proteoglycans) and/or LRP (low density lipoprotein receptor related protein) (Ananyeva et al. 2001, TCM, 11:251-257. A suitable test to determine the procoagulant activity of Factor VIII is the one stage or the two stage coagulation assay (Rizza et al. 1982 Coagulation assay of FVIIIc and FIXa in Bloom ed. The Hemophilias. NY Churchchill Livingston 1992).

The cDNA sequence and the amino acid sequence of the mature wild type form of human blood coagulation Factor VIII are shown in SEQ ID NO:1 and SEQ ID NO:2, respectively. The reference to an amino acid position of a specific sequence does not exclude the presence of mutations, e.g. deletions, insertions and/or substitutions at other positions in the sequence referred to. For example, a mutation in “Glu2004” referring to SEQ ID NO:2 does not exclude that in the modified homologue one or more amino acids at positions 1 through 2003 of SEQ ID NO:2 are missing.

The modified FVIII homologue of the invention exhibits an increased functional half-life after thrombin activation compared to the non-modified form and/or to the wild type form FVIII. The functional half-life can be determined in vitro as shown in FIG. 5 of US 2003/0125232 or as published by Sandberg (Thromb. Haemost. 2001; 85(1):93-100) and Gale (Gale et al., J. Thromb. Haemost., 2003, 1: p. 1966-1971) which basically consists of determining the kinetics of FVIII activity after thrombin activation. In vivo one could test the increased functional half-life of the modified FVIII in animal models of hemophilia A, like FVIII knockout mice, in which one would expect a longer lasting hemostatic effect of a stabilized FVIII or a higher hemostatic effect at the same concentration as compared to wild type FVIII. The hemostatic effect could be tested for example by determining time to arrest of bleeding after a tail clip.

The modified FVIII variants of this invention retain 40 minutes after activation by thrombin more than 25%, or more preferred more than 50% or even more preferred more than 75% of their initial peak activity as measured in vitro.

The functional half life is usually increased by at least 50%, preferably by at least 100%, more preferably by at least 200%, even more preferably by at least 500% compared to the non-modified form and/or to the wild type form of the modified FVIII variant.

The functional half-life of the wild type form of human Factor VIIIa is 2.1 minutes. The functional half life of the modified Factor VIIIa molecule of the invention is usually at least about 3.15 minutes, preferably at least about 4.2 minutes, more preferably at least about 6.3 minutes, most preferably at least about 12.6 minutes.

The invention further relates to a polynucleotide encoding a modified human FVIII variant as described in this application. The term “polynucleotide(s)” generally refers to any polyribonucleotide or polydeoxyribonucleotide that may be unmodified RNA or DNA or modified RNA or DNA. The polynucleotide may be single- or double-stranded DNA, single or double-stranded RNA. As used herein, the term “polynucleotide(s)” also includes DNAs or RNAs that comprise one or more modified bases and/or unusual bases, such as inosine. It will be appreciated that a variety of modifications may be made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells.

The skilled person will understand that, due to the degeneracy of the genetic code, a given polypeptide can be encoded by different polynucleotides. These “variants” are encompassed by this invention.

“Factor VIII” as used in this application means a product consisting of the non-activated form (Factor VIII). “Factor VIII” within the above definition includes proteins that have the amino acid sequence of native human Factor. VIII. It also includes proteins with a slightly modified amino acid sequence, for instance, a modified N-terminal end including N-terminal amino acid deletions or additions so long as those proteins substantially retain the activity of Factor Villa. “Factor VIII” within the above definition also includes natural allelic variations that may exist and occur from one individual to another. “Factor VIII” within the above definition further includes variants of FVIII. Such variants differ in one or more amino acid residues from the wild type sequence. Examples of such differences may include truncation of the N- and/or C-terminus by one or more amino acid residues (e.g. 1 to 10 amino acid residues), or addition of one or more extra residues at the N- and/or C-terminus, e.g. addition of a methionine residue at the N-terminus, as well as conservative amino acid substitutions, i.e. substitutions performed within groups of amino acids with similar characteristics, e.g. (1) small amino acids, (2) acidic amino acids, (3) polar amino acids, (4) basic amino acids, (5) hydrophobic amino acids, (6) aromatic amino acids and (7) polar amino acids. Examples of such conservative substitutions are shown in the following table.

| (1) | Alanine | Glycine | | |
|---|---|---|---|---|
| (2) | Aspartic acid | Glutamic acid | | |
| (3) | Asparagine | Glutamine | | |
| (4) | Arginine | Histidine | Lysine | |
| (5) | Isoleucine | Leucine | Methionine | Valine |
| (6) | Phenylalanine | Tyrosine | Tryptophane | |
| 7 | Serine | Threonine | | |

Preferably, the polynucleotide of the invention is an isolated polynucleotide. The term “isolated” polynucleotide refers to a polynucleotide that is substantially free from other nucleic acid sequences, such as and not limited to other chromosomal and extrachromosomal DNA and RNA. Isolated polynucleotides may be purified from a host cell. Conventional nucleic acid purification methods known to skilled artisans may be used to obtain isolated polynucleotides. The term also includes recombinant polynucleotides and chemically synthesized polynucleotides.

Yet another aspect of the invention is a plasmid or vector comprising a polynucleotide according to the invention. Preferably, the plasmid or vector is an expression vector. In a particular embodiment, the vector is a transfer vector for use in human gene therapy.

Still another aspect of the invention is a host cell comprising a polynucleotide of the invention or a plasmid or vector of the invention.

The host cells of the invention may be employed in a method of producing a modified FVIII variant, which is part of this invention. The method comprises:
(a) culturing host cells of the invention under conditions such that the modified FVIII variant is expressed; and
(b) optionally recovering the modified FVIII variant from the host cells or from the culture medium.

Degree and location of glycosylation or other post-translation modifications may vary depending on the chosen host cells and the nature of the host cellular environment. When referring to specific amino acid sequences, posttranslational modifications of such sequences are encompassed in this application.

It is preferred to purify the modified homologue of the present invention to ≧80% purity, more preferably ≧95% purity, and particularly preferred is a pharmaceutically pure state that is greater than 99.9% pure with respect to contaminating macromolecules, particularly other proteins and nucleic acids, and free of infectious and pyrogenic agents. Preferably, an isolated or purified modified homologue of the invention is substantially free of other polypeptides.

The various products of the invention are useful as medicaments. Accordingly, the invention relates to a pharmaceutical composition comprising a modified FVIII variant as described herein, a polynucleotide of the invention, or a plasmid or vector of the invention.

The recombinant proteins described in this invention can be formulated into pharmaceutical preparations for therapeutic use. The purified proteins may be dissolved in conventional physiologically compatible aqueous buffer solutions to which there may be added, optionally, pharmaceutical excipients to provide pharmaceutical preparations.

Such pharmaceutical carriers and excipients as well as suitable pharmaceutical formulations are well known in the art (see for example “Pharmaceutical Formulation Development of Peptides and Proteins”, Frokjaer et al., Taylor & Francis (2000) or “Handbook of Pharmaceutical Excipients”, 3^{rd} edition, Kibbe et al., Pharmaceutical Press (2000)). In particular, the pharmaceutical composition comprising the polypeptide variant of the invention may be formulated in lyophilized or stable soluble form. The polypeptide variant may be lyophilized by a variety of procedures known in the art. Lyophilized formulations are reconstituted prior to use by the addition of one or more pharmaceutically acceptable diluents such as sterile water for injection or sterile physiological saline solution.

Formulations of the composition are delivered to the individual by any pharmaceutically suitable, means of administration. Various delivery systems are known an can be used to administer the composition by any convenient route. Preferentially the compositions of the invention are administered systemically. For systemic use, the FVIII variants of the invention are formulated for parenteral (e.g. intravenous, subcutaneous, intramuscular, intraperitoneal, intracerebral, intrapulmonar, intranasal or transdermal) or enteral (e.g., oral, vaginal or rectal) delivery according to conventional methods. The most preferential route of administration is intravenous administration. The formulations can be administered continuously by infusion or by bolus injection. Some formulations encompass slow release systems.

The modified biologically active FVIII variants of the present invention are administered to patients in a therapeutically effective dose, meaning a dose that is sufficient to produce the desired effects, preventing or lessening the severity or spread of the condition or indication being treated without reaching a dose which produces intolerable adverse side effects. The exact dose depends on many factors as e.g. the indication, formulation, mode of administration and has to be determined in preclinical and clinical trials for each respective indication.

The pharmaceutical composition of the invention may be administered alone or in conjunction with other therapeutic agents. These agents may be incorporated as part of the same pharmaceutical.

Another aspect of the invention is the use of a modified FVIII variant as described herein, of a polynucleotide of the invention, of a plasmid or vector of the invention, or of a host cell of the invention for the manufacture of a medicament for the treatment or prevention of a blood coagulation disorder. Blood coagulation disorders include but are not limited to hemophilia A. Preferably, the treatment comprises human gene therapy.

The invention also concerns a method of treating an individual suffering from a blood coagulation disorder such as hemophilia A. The method comprises administering to said individual an efficient amount of the modified FVIII variant as described herein. In another embodiment, the method comprises administering to the individual an efficient amount of the polynucleotide of the invention or of a plasmid or vector of the invention. Alternatively, the method may comprise administering to the individual an efficient amount of the host cells of the invention described herein.

### Expression of the Proposed Mutants

The production of recombinant mutant proteins at high levels in suitable host cells requires the assembly of the above-mentioned modified cDNAs into efficient transcriptional units together with suitable regulatory elements in a recombinant expression vector that can be propagated in various expression systems according to methods known to those skilled in the art. Efficient transcriptional regulatory elements could be derived from viruses having animal cells as their natural hosts or from the chromosomal DNA of animal cells. Preferably, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA transcribed from the cDNAs, the transcriptional unit should contain in its 3′-proximal part a DNA region encoding a transcriptional termination-polyadenylation sequence. Preferably, this sequence is derived from the Simian Virus 40 early transcriptional region, the rabbit beta-globin gene, or the human tissue plasminogen activator gene.

The cDNAs are then integrated into the genome of a suitable host cell line for expression of the Factor. VIII proteins. Preferably this cell line should be an animal cell-line of vertebrate origin in order to ensure correct folding, disulfide bond formation, asparagine-linked glycosylation and other post-translational modifications as well as secretion into the cultivation medium. Examples on other post-translational modifications are tyrosine O-sulfation, and proteolytic processing of the nascent polypeptide chain. Examples of cell lines that can be use are monkey COS-cells, mouse L-cells, mouse C127-cells, hamster BHK-21 cells, human embryonic kidney 293 cells, and preferentially hamster CHO-cells.

The recombinant expression vector encoding the corresponding cDNAs can be introduced into an animal cell line in several different ways. For instance, recombinant expression vectors can be created from vectors based on different animal viruses. Examples of these are vectors based on baculovirus, vaccinia virus, adenovirus, and preferably bovine papilloma virus.

The transcription units encoding the corresponding DNA's can also be introduced into animal cells together with another recombinant gene which may function as a dominant selectable marker in these cells in order to facilitate the isolation of specific cell clones which have integrated the recombinant DNA into their genome. Examples of this type of dominant selectable marker genes are Tn5 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated to the genome of the host cell, frequently resulting in a tight physical linkage between the different transcription units.

Other types of selectable marker genes which can be used together with the cDNA of the desired protein are based on various transcription units encoding dihydrofolate reductase (dhfr). After introduction of this type of gene into cells lacking endogenous dhfr-activity, preferentially CHO-cells (DUKX-B11, DG-44) it will enable these to grow in media lacking nucleosides. An example of such a medium is Ham's F12 without hypoxanthine, thymidin, and glycine. These dhfr-genes can be introduced together with the Factor VIII cDNA transcriptional units into CHO-cells of the above type, either linked on the same vector or on different vectors, thus creating dhfr-positive cell lines producing recombinant protein.

If the above cell lines are grown in the presence of the cytotoxic dhfr-inhibitor methotrexate, new cell lines resistant to methotrexate will emerge. These cell lines may produce recombinant protein at an increased rate due to the amplified number of linked dhfr and the desired protein's transcriptional units. When propagating these cell lines in increasing concentrations of methotrexate (1-10000 nM), new cell lines can be obtained which produce the desired protein at very high rate.

The above cell lines producing the desired protein can be grown on a large scale, either in suspension culture or on various solid supports. Examples of these supports are micro carriers based on dextran or collagen matrices, or solid supports in the form of hollow fibres or various ceramic materials. When grown in cell suspension culture or on micro carriers the culture of the above cell lines can be performed either as a bath culture or as a perfusion culture with continuous production of conditioned medium over extended periods of time. Thus, according to the present invention, the above cell lines are well suited for the development of an industrial process for the production of the desired recombinant mutant proteins

The recombinant mutant protein, which accumulates in the medium of secreting cells of the above types, can be concentrated and purified by a variety of biochemical and chromatographic methods, including methods utilizing differences in size, charge, hydrophobicity, solubility, specific affinity, etc. between the desired protein and other substances in the cell cultivation medium.

An example of such purification is the adsorption of the recombinant mutant protein to a monoclonal antibody which is immobilised on a solid support. After desorption, the protein can be further purified by a variety of chromatographic techniques based on the above properties.

The recombinant proteins described in this invention can be formulated into pharmaceutical preparations for therapeutic use. The purified proteins may be dissolved in conventional physiologically compatible aqueous buffer solutions to which there may be added, optionally, pharmaceutical excipients to provide pharmaceutical preparations.

The modified polynucleotides (e.g. DNA) of this invention may also be integrated into a transfer vector for use in the human gene therapy.

The various embodiments described herein may be combined with each other. The present invention will be further described more in detail in the following examples thereof. This description of specific embodiments of the invention will be made in conjunction with the appended figures.

### DESCRIPTION OF THE DRAWINGS

FIG. 1a:

Nucleotide and amino acid sequence of the linker insertion site within the FVIII coding sequence.

FIG. 1b:

Amino acid sequences of linkers. Gly374 (underlined) is replaced by the linker sequences indicated.

FIG. 2:

Determination of FVIII antigen production (Panel A) and FVIII specific activity (Panel B) from COS cells supernatants following transient transfection. COS cells (4×10⁵ cells per well) were transfected using 5 μl FuGENE 6™ (Roche Diagnostics, Meylan, France) preincubated with 1 μg of plasmid DNA. Two days after transfection, COS cells were washed and placed in Iscove's modified Dulbecco medium (IMDM) supplemented with 1% BSA. The conditioned media were harvested after 6 hours. FVIII antigen was quantified using an ELISA kit (Diagnostica Stago, Asmières, France) and FVIII activity was measured using 2 methods: a chromogenic method (“two-stages clotting assay” Coamatic FVIII, Chromogenix, Milano, Italy) or a chronometric method (“one-stage clotting assay”).

FIG. 3:

Immunoblot analysis of heparin-purified FVIII L99. Heparin-purified FVIII and ReFacto were diluted in Hepes 20 mM pH 7.4, CaCl₂ 5 mM, Tween 20 0.01% in order to obtain a final concentration of 2250 ng/ml. Samples (50 μl) were then diluted in Laemmli buffer (25 μl), boiled and analyzed on SDS-PAGE. 20 μl of each sample were loaded per lane. FVIII was detected using a mixture of 2 mouse antibodies: an anti-light chain and an anti-heavy chain.

FIG. 4:

Immunoblot analysis of heparin-purified FVIII WT and FVIII L99 following their activation by thrombin. Heparin-purified FVIII WT and L99 were diluted in IMDM in the presence of 5 mM CaCl₂ and 2.5% glycerol. Each FVIII was activated at 37° C. by thrombin (1 U FVIII/1 U Thrombin) during different, incubation times. The reaction was blocked using hirudin (1 U FVIII/2 U Hirudin), immediately diluted in Laemmli buffer and boiled. The samples corresponding to 26 ng of FVIII were then submitted to immunoblotting and detected with the mixture of the anti-light chain and the anti-heavy chain antibodies.

FIG. 5:

Comparison of FVIIIa inactivation kinetics after thrombin activation of FVIII WT and FVIII L99. FXa generation was realized using 50 ng of FVIII antigen in 150 μl final volume at 37° C. FXa generation was made in a buffer containing 150 mM NaCl, 20 mM Hepes pH 7.4 and 5 mM CaCL₂. 2 μM PC/PS 75/25 and 0.5% BSA. The revelation mix contains 93 nM FX, 1 nM FIXa and 0.5 mM Spectrozyme.

FIG. 6:

HuAPC inactivation kinetics of activated FVIII L99. 50 ng of FVIII were used for this test. Two ratios of FVIII/APC were used: ratio 1/1 (Panel A) or 1/6 (Panel B). For each ratio, various concentrations of protein S were assayed. The tables summarized the ratios used for each molecule.

### EXAMPLES

### Example 1

### Generation of Factor VIII Mutants

Basis for introduction of mutations into the FVIII cDNA sequence was a B domain deleted FVIII sequence containing truncated FIX introns (Plantier J L et al. Thromb. Haemost. 86:596-603 (2001)). The FVIII sequence was transferred from pcDNA3.1 into pKSII+ (Stratagene) through a NotI/XhoI fragment resulting in plasmid pKS-174. Deletion of the thrombin cleavage site at position 372 was achieved by changing Arg372 into Ala by site directed mutagenesis, using standard methods (QuickChange XL Site Directed Mutagenesis Kit, Stratagene) and oligonucleotides We1013 and We1014 (SEQ ID NO 3 and 4). To introduce a restriction site for insertion of GlySer linker coding sequences, the resulting plasmid was subjected to another round of mutagenesis using oligonucleotides We1015 and We1016 (SEQ ID NO 5 and 6) changing Val374 into Gly and thereby creating a new NarI site. The resulting plasmid was designated pKS-190.

Linker modules providing various restriction sites for linker concatemerization and insertion into the respective plasmid, respectively, were first cloned into pCR4Topo vector (Invitrogen). 5 overlapping oligonucleotide pairs, We884/We1052 (fragment 1, SEQ ID NO 7 and 8), We884/We1053 (fragment 2; SEQ ID NO 7 and 9), We1051/We1052 (fragment 3; SEQ ID NO 10 and 8), We1051/We1054 (fragment 4; SEQ ID NO 10 and 11) and We890/We1052 (fragment 5; SEQ ID NO 12 and 8) were each annealed, elongated and amplified by PCR to generate the 5 linker fragments. For this purpose 10 pmoles of each oligonucleotide pair were PCR amplified by an initial denaturation at 95° C. for 2 minutes, 10 thermocycles of 15 seconds at 94° C., 15 seconds at 55° C. and 15 seconds at 72° C., followed by a final extension at 72° C. for 3 minutes. Each fragment was then cloned into pCR4Topo. Subsequently fragments were excised from the vectors by digest with respective restriction endonucleases. Fragment 1 was excised by MspI/NarI, fragment 2 by MspI/BamH1, fragment 3 by BgIII/NarI, fragment 4 by BgIII/BspEI and fragment 5 by BspEII/NarI, followed by gel purification using standard methods (Qiagen).

For insertion of the linker fragments into the FVIII sequence, plasmid pKS-190 was linearized with NarI and linker fragments and combinations thereof were inserted. To insert a 20 mer linker, fragment 1 was used, the resulting plasmid was designated pKS-249. To insert a 42 mer linker, fragments 2 and 3 were combined, the resulting plasmid was designated pKS-250. To insert a 61 mer linker, fragments 2, 4 and 5 were combined, the resulting plasmid was designated pKS-251. The insertion of one and 2 copies of fragment 1, respectively, into NarI linearized plasmid pKS-251 resulted in plasmid pKS-259 containing a 80 mer linker and plasmid pKS-260 containing a 99 mer linker. The insertion of fragment 1 into the NarI site of plasmid 260 yielded plasmid pKS-279 containing a 118 mer linker. Insertion of fragments 2 and 3 into the NarI site of plasmid 260 yielded plasmid pKS-280 containing a 140 mer linker. And insertion of fragments 2, 4 and 5 into the NarI site of plasmid 260 yielded plasmid pKS-281 containing a 159 mer linker.

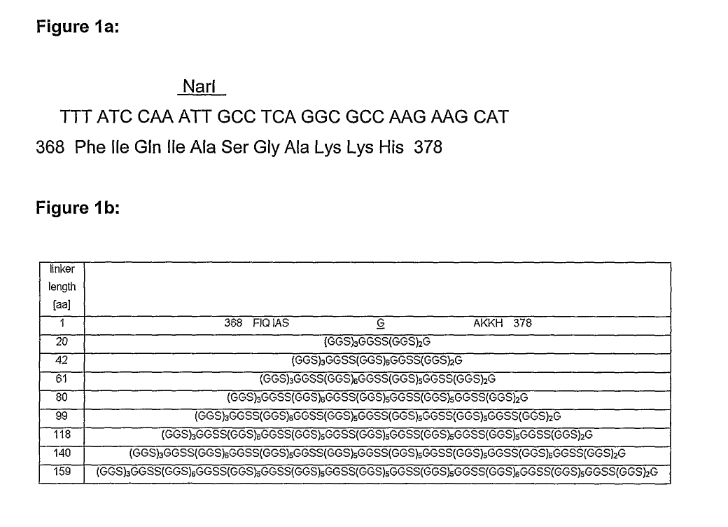

After sequence verification of the linker inserts the FVIII sequences containing the linkers of various lengths were transferred back into expression vector pcDNA3.1 through their NotI/XhoI sites. Table 1 summarizes linker length and plasmid numbers in pKSII+ and pcDNA3.1. FIG. 1 illustrates the amino acid sequences of the various linkers in the FVIII context.

TABLE 1

| linker | plasmid in | plasmid in | designation of |
|---|---|---|---|
| length [aa] | pKSII+ | pcDNA3.1 | FVIII protein * |
| 1 | pKS-190 | pcDNA3-252 | L0 |
| 20 | pKS-249 | pcDNA3-253 | L20 |
| 42 | pKS-250 | pcDNA3-254 | L42 |
| 61 | pKS-251 | pcDNA3-255 | L61 |
| 80 | pKS-259 | pcDNA3-261 | L80 |
| 99 | pKS-260 | pcDNA3-262 | L99 |
| 118 | pKS-279 | pcDNA3-282 | L118 |
| 159 | pKS-281 | pcDNA3-284 | L159 |

* all proteins carry the R372A mutation and a deletion of V374

### Example 2

### Expression of Factor VIII Mutants

Transfection of Factor VIII mutant clones and expression of the mutant Factor VIII molecules is done as described previously and known to those skilled in the art (e.g. Plantier J L et al. Thromb. Haemost. 86:596-603 (2001)).

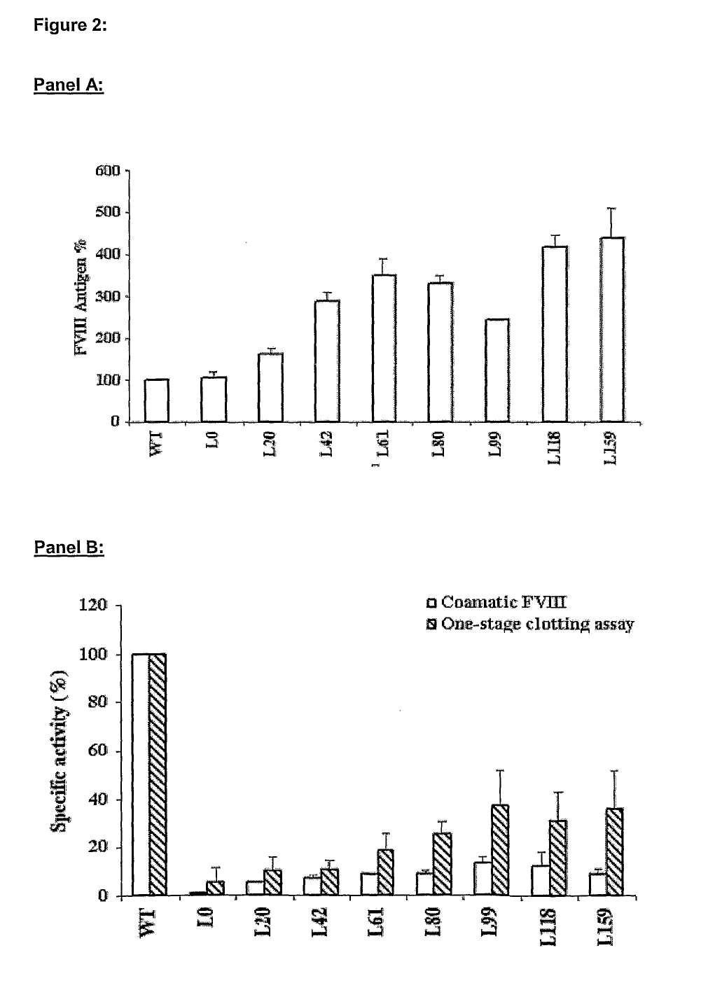

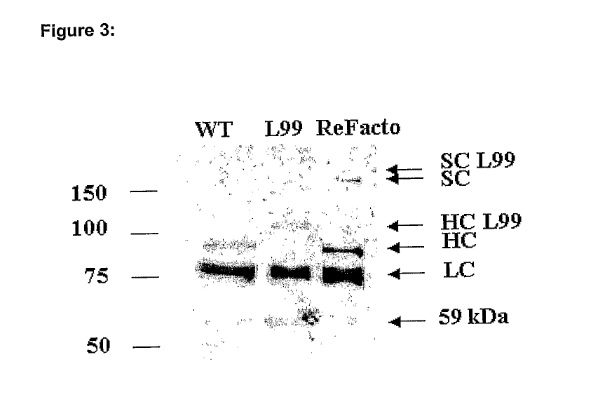

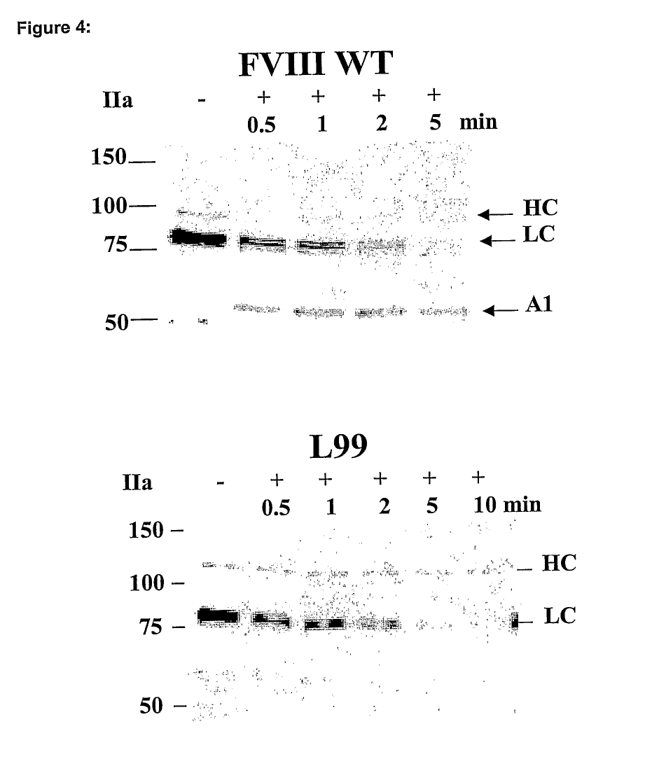

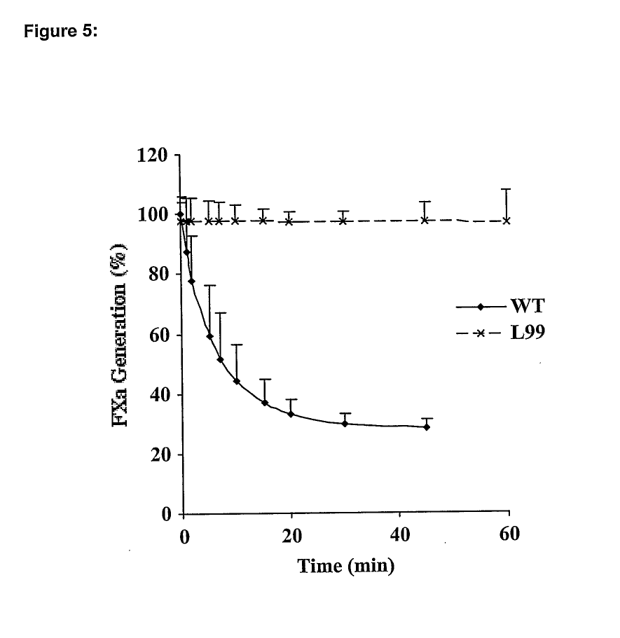

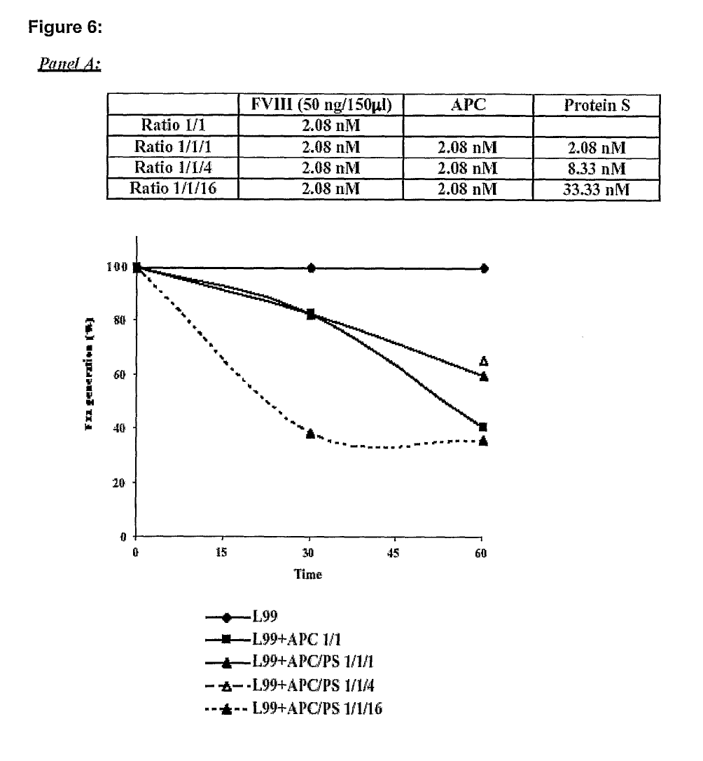

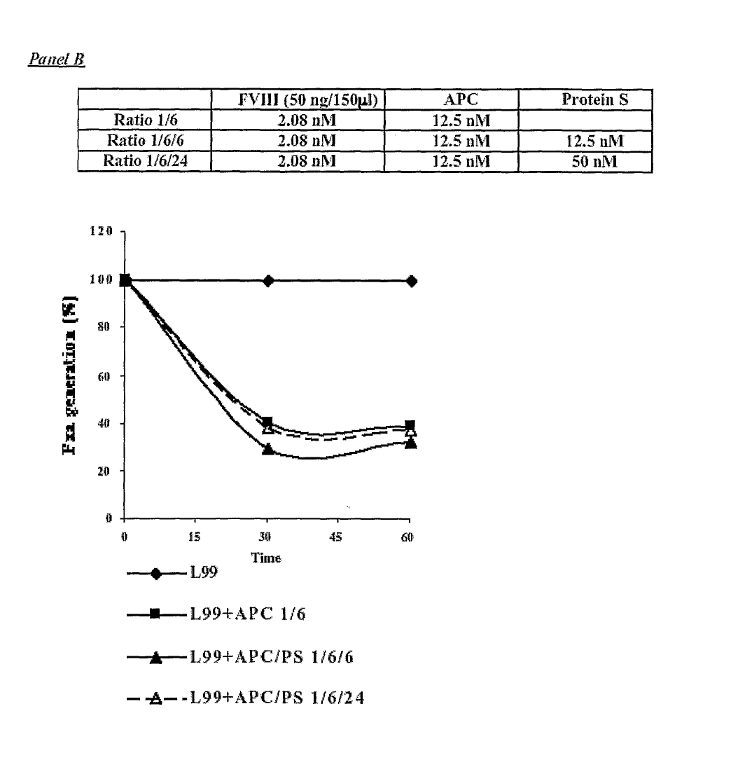

Following transfection in COS cells, all FVIII mutants were produced and secreted in the medium in similar or even higher amounts than FVIII WT (FIG. 2, Panel A). No FVIII activity was detected from the supernatants of L0 expressing COS cells which is an expected result since the R372A mutation is responsible for a severe hemophilia A. Using Coamatic FVIII assay (“two-stage clotting assay”), FVIII activities from mutants were low compared to the activity obtained with FVIII WT. However, the activity regularly increased with the length of the linker. The highest specific activity was obtained with. FVIII. L99 mutant (around 13% of the control) and was not increased with mutants bearing longer linkers. Using the chronometric assay (“one-stage clotting assay”), FVIII activity also increased with the length of the linker. However, the specific activity reach a much higher levels than obtained with the two-stage clotting assay attaining up to 37% of the control activity. Using this second technique also, the highest specific activity was obtained with FVIII L99 (FIG. 2, Panel B).

In summary, FVIII L99 was efficiently produced in COS cells and presented the highest specific activities obtained from all the linker mutants. Therefore to further characterize this molecule, the FVIII L99 construct was stably transfected in CHO cells.

### Example 3

### Functional Analysis of FVIII L99

### Heparin Chromatography

FVIII L99 was produced using roller bottles and, purified using heparin chromatography. FVIII activity of the semi-purified FVIII L99 was quantified using coamatic FVIII or “one-stage clotting assay”. The discrepancy between one-stage and two-stage clotting assay seen in COS supernatants was also seen in CHO cells. Compared to semi-purified FVIII WT (100%), the specific activity measured with coamatic FVIII was low (7% of the control; n=3 purifications) whereas the specific activity obtained with the “one-stage clotting assay” was higher than FVIII WT (195% of the control (FVIII WT); n=3 purifications).

Semi-purified proteins were further studied using western blot analysis. The detection of the proteins was realized using a mixture of 2 antibodies: an anti-light chain (aLC) and an anti-heavy chain (aHC) antibodies using the ECL system (Amersham Biosciences, Orsay, France The anti-HC antibody specifically detected the A1 chain (FIG. 3).

In these reducing conditions, L4 and ReFacto FVIII have similar migration profiles. The L99 has a LC with a molecular mass similar to the control FVIII LC. However, the migration of its HC was different than controls due to the presence of the linker that increased its molecular mass. A 59 kDa supplementary band was detected in all the tested samples.

### Example 4

### Thrombin Activation

Heparin-purified FVIII was thereafter activated with thrombin. The reaction was realized in the presence of CaCl₂ (5 mM) and glycerol (2.5%) in Iscove's modified Dulbecco medium (IMDM).

Each FVIII aliquot (98 ng per time point) was activated by 0.49 U of thrombin during different incubation times. The reaction was blocked using hirudin (0.98 U) and then immediately diluted in Laemmli Buffer. The samples were submitted to immunoblotting.

The A1 chain of the FVIII WT was clearly detected after a 30 sec incubation time with thrombin, confirming the expected cleavage of the HC. The signal corresponding to the LC totally disappeared following 5 min of thrombin activation. Thrombin is known to cleave the Arg1689, liberating the a3 domain. This result suggested that the epitope of the anti-LC antibody seems to be within the a3 domain and, that the LC domain is totally cleaved following 5 min of thrombin activation. After 5 min in the presence of thrombin, both the HC and the LC of FVIII WT were demonstrated to be totally activated.

In the case of the FVIII L99, its LC was identically cleaved as FVIII WT (i.e. disappearing after 5 min of thrombin incubation). In contrast, as expected the migration profile of its HC remained unmodified following thrombin activation. Furthermore, no A1 domain signal could be shown even with longer ECL revelation times. This result demonstrated that the cleavage between A1 and A2 was prevented (FIG. 4).

### Example 5

### Stability after Thrombin Activation of FVIII L99

The study of the half-life of thrombin activated WT-FVIII or L99 was realized using the FXa generation assay. The test was realized using 50 ng of FVIII antigen. Each FVIII was activated for 2 min by thrombin and the FVIIIa remaining activity measured at different time points. The determination of activated FVIII WT or L99 half-life was realized using the FXa generation assay. The test was performed at 37° C. using 50 ng of FVIII antigen in 150 μl final volume. FXa generation was made in a buffer containing 150 mM NaCl, 20 mM Hepes pH 7.4 and 5 mM CaCL₂, 2 μM PC/PS 75/25 and 0.5% BSA. Each FVIII was activated for 2 min by thrombin. The reaction was then blocked by hirudin (1 U FVIII/1 U thrombin/2 U hirudin). FVIIIa remaining activity was thereafter measured at different time point by addition of a revelation mix containing 93 nM FX, 1 nM FIXa and 0.5 mM Spectrozyme. The appearance of colored products was monitored at 405 nm.

The FVIII activity from the FVIII WT decreased rapidly. The half-life of activated FVIII WT was found to be around 4.69 min. In the case of L99, its activity remained roughly stable following thrombin activation and showed no decrease during the 1 hour incubation time (FIG. 5).

### Example 6

### APC Inactivation of the Activated FVIII L99

APC inactivation with or without protein S (Protein S: Diagnostica Stago, Asnières, France, hAPC: Aventis Behring, Marburg, Germany) was tested on activated heparin-purified FVIII L99. 50 ng of FVIII L99 were activated with thrombin during 2 min before the addition of the human APC with or without protein S At different time points, the remaining FVIII activity was detected with the FXa generation test.

These results demonstrated that FVIII L99 could be inactivated by human APC with or without Protein S. When the ratio FVIII/APC was decreased (1/6), the inactivation was already at its maximum using the single APC and the addition of protein S did not further diminished FVIII activity.

### Summary of the Results:

Several FVIII mutants characterized by the insertion of different peptidic linkers substituting the thrombin activation site at Arg372 were generated. These modified FVIII were well expressed after COS cell transfection. Whereas FVIII L0 did not show FVIII procoagulant activity, FVIII mutants bearing a linker do have one. The level of this activity increased concomitantly with the length of the linker reaching a maximum when 99 amino acids were inserted. Using the chronometric method, the FVIII activity detected with FVIII L99 was similar to FVIII WT whereas. FVIII L118 and FVIII L159 demonstrated no further improvement of the molecule.

Heparin-purified L99 showed a discrepancy between “one-stage” and “two stage” clotting assay that remained unexplained until now. However, immunoblot analysis demonstrated thrombin activation kinetics similar to FVIII WT and the specific activity, when measured with the chronometric method, was even higher than FVIII WT. Interestingly, activated FVIII L99 was almost stable during more than 1 hour. Finally, APC recognized this modified FVIII and was able to efficiently inactivate the FVIII L99.

1. A modified recombinant Factor VIII (FVIII) variant which is biologically active after thrombin activation with improved stability of its activated form, wherein said modified recombinant FVIII is modified so that thrombin cleavage between the A1 and the A2 domain of FVIII is prevented and the A2 domain remains covalently attached to the A1 domain after thrombin activation.

2. The modified biologically active recombinant FVIII variant according to claim 1, wherein the A2 domain is covalently linked to the A1 domain through a peptidic linker which is not cleavable by thrombin.

3. The modified biologically active recombinant FVIII variant according to claim 2 wherein the peptidic linker consists of repeats of the amino acids Gly and Ser.

4. The modified biologically active recombinant FVIII variant according to claim 2 wherein the peptidic linker consists of 80 to 120 amino acids.

5. The modified biologically active recombinant FVIII variant according to claim 2 wherein the peptidic linker consists of 90 to 110 amino acids.

6. The modified biologically active recombinant FVIII variant according to claim 2 wherein the peptidic linker consists of 99 amino acids.

7. The modified biologically active recombinant FVIII variant according to claim 1, wherein said FVIII variants has a functional half-life increased by at least 50% compared to FVIII wild type.

8. The modified biologically active recombinant FVIII variant according to claim 1, which retains more than 25% of its initial peak activity for at least about 40 minutes after activation by thrombin.

9. The modified biologically active recombinant FVIII variant according to claim 1, wherein mutations are inserted either in the wild-type FVIII or in a FVIII in which a B-domain is partially or completely deleted and may be replaced by a linker.

10. A polynucleotide encoding the modified FVIII variant according to claim 1.

11. A plasmid or vector comprising the polynucleotide according to claim 10.

12. The plasmid or vector according to claim 11, which is an expression vector.

13. The plasmid or vector according to claim 11, which is a transfer vector for use in human gene therapy.

14. A host cell comprising the polynucleotide according to claim 10.

15. A method of producing a modified FVIII variant according to claim 1, comprising:
a. culturing host cells according to claim 14 under conditions such that the modified FVIII variant is expressed; and
b. optionally recovering the modified FVIII variant from the host cells or from the culture medium.

16. A pharmaceutical composition comprising a modified FVIII according to claim 1 and a pharmacologically acceptable carrier.

17. A method of treating or preventing a blood coagulation disorder, wherein said method comprises administering an effective amount of an agent selected from
a. a modified recombinant Factor VIII (FVIII) variant which is biologically active after thrombin activation with improved stability of its activated form, wherein said modified recombinant FVIII is modified in a way that thrombin cleavage between the A1 and the A2 domain of FVIII is prevented and the A2 domain remains covalently attached to the A1 domain after thrombin activation;
b. a polynucleotide encoding the modified FVIII variant according to subpart (a);
c. a plasmid or vector comprising the polynucleotide according to subpart (b);
d. a host cell comprising the polynucleotide according to subpart (b); and
e. a host cell comprising the plasmid or vector according to subpart (c).

18. The method according to claim 17, wherein the blood coagulation disorder is hemophilia A.

19. The method according to claim 17, wherein the method comprises human gene therapy.

20. The modified biologically active recombinant FVIII variant according to claim 9, the B-domain is partially or completely replaced by a linker.

21. A host cell comprising the plasmid or vector according to claim 11.

22. A pharmaceutical composition comprising a polynucleotide according to claim 10 and a pharmacologically acceptable carrier.

23. A pharmaceutical composition comprising a plasmid or vector according to claim 11 and a pharmacologically acceptable carrier.

24. The method according to claim 17, wherein the agent is a modified recombinant Factor VIII (FVIII) variant which is biologically active after thrombin activation with improved stability of its activated form, wherein said modified recombinant FVIII is modified in a way that thrombin cleavage between the A1 and the A2 domain of FVIII is prevented and the A2 domain remains covalently attached to the A1 domain after thrombin activation.

25. The method according to claim 19, wherein the blood coagulation disorder is hemophilia A.


SEQUENCE LISTING (12 sequences)

SEQ ID NO: 1 - DNA, 7056 bp, Homo sapiens
  misc_feature (58)..(7053): Bases from 58 to 7053 are a CDS for mature human Factor VIII
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 2 - protein, 2332 aa, Homo sapiens
  mat_peptide (1)..(2332)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHRSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - DNA, 37 bp, artificial
  artificial
ccttcctttatccaaattgcctcagttgccaagaagc

SEQ ID NO: 4 - DNA, 37 bp, artificial
  artificial
gcttcttggcaactgaggcaatttggataaaggaagg

SEQ ID NO: 5 - DNA, 36 bp, artificial
  artificial
ctttatccaaattgcctcaggcgccaagaagcatcc

SEQ ID NO: 6 - DNA, 36 bp, artificial
  artificial
ggatgcttcttggcgcctgaggcaatttggataaag

SEQ ID NO: 7 - DNA, 44 bp, artificial
  artificial
gtgccgggtcgggtggtagtggggggagcggcggctcttctggc

SEQ ID NO: 8 - DNA, 45 bp, artificial
  artificial
cacggcgcccgatcctccactgccgccagaagagccgccgctccc

SEQ ID NO: 9 - DNA, 48 bp, artificial
  artificial
cacggatccgcccgatcctccactgccgccagaagagccgccgctccc

SEQ ID NO: 10 - DNA, 51 bp, artificial
  artificial
gtgagatctggcgggtcgggtggtagtggggggagcggcggctcttctggc

SEQ ID NO: 11 - DNA, 42 bp, artificial
  artificial
cactccggaacctccactgccgccagaagagccgccgctccc

SEQ ID NO: 12 - DNA, 48 bp, artificial
  artificial
gtgtccggagggtcgggtggtagtggggggagcggcggctcttctggc